(19) United States
(12) Patent Application Publication
(10) Pub. No.: US 2004/0109870 A1
(43) Pub. Date: Jun. 10, 2004

(54) Therapeutic agent for acute hepatitis and chronic hepatitis including hepatic fibrosis and cirrhosis

(76) Inventors: Junji Yodoi, Kyoto-shi (JP); Hajime Nakamura, Hirakata-shi (JP); Hiroaki Okuyama, Yao-shi (JP); Yasuyuki Shimahara, Kyoto-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/457,890

(22) Filed: Jun. 10, 2003

(30) Foreign Application Priority Data
Aug. 2, 2002 (JP) 2002-226552

(51) Int. Cl. A61K 39/00

(52) U.S. Cl. 424/185.1

(57) ABSTRACT

The present invention relates to a therapeutic agent for treating acute hepatitis and chronic hepatitis (including hepatic fibrosis and hepatic cirrhosis) comprising as active ingredient one or more polypeptide or polypeptides selected from the polypeptides belonging to the family having thioredoxin activity.

### TECHNICAL FIELD

The present invention relates to a prophylactic or therapeutic agent for acute hepatitis and for chronic hepatitis including chronic hepatic fibrosis and hepatic cirrhosis.

### BACKGROUND OF THE INVENTION

Thioredoxin (TRX) is a small multifunctional protein with a molecular weight of 12 kDa, containing a redox-active disulfide/dithiol within the active site sequence: -Cys-Gly-Pro-Cys- (Nakamura H., Nakamura K., Yodoi J., Redox regulation of cellular activation., Annu. Rev. Immunol.; 1997, 15: 351-369). Thioredoxin was first isolated from Escherichia coli as a hydrogen donor for ribonucleotide reductase, an important enzyme for the synthesis of deoxyribonucleotides. Since then thioredoxin has been isolated and identified from a variety of prokaryotes and eukaryotes. Adult T cell leukemia-derived factor (ADF) is a human thioredoxin first identified by the present inventors as an IL-2 receptor inducing factor produced by HTLV-I-transfected T-lymphocytes. Intracellular thioredoxin plays an important role in scavenging radicals and regulating redox-related transcription factors, such as Activator Protein-1 and Nuclear Factor-κB (Hirota K., Matsui M., Iwata S., Nishiyama A., Mori K., Yodoi J., AP-1 transcriptional activity is regulated by a direct association between thioredoxin and Ref-1., Proc. Natl. Acad. Sci. USA; 1997, 94(8): 3633-3638). In addition, human thioredoxin controls the signal transductions of p38 mitogen activating protein kinase (MAPK) and apoptosis signal-regulating kinase 1 (ASK-1). Human thioredoxin shows resistance to CDDP-induced cytotoxicity. The present inventors have shown that TRX transgenic mice (TRX Tg mice) are resistant to cerebral ischemia, influenza virus pneumonia and other diseases caused by oxidative stress (Takagi Y., Mitsui A., Nishiyama A., et al., Overexpression of thioredoxin in transgenic mice attenuates focal ischemic brain damage., Proc. Natl. Acad. Sci. USA; 1999, 96(7): 4131-4136).

Oxidative stress is a significant cause of hepatic diseases (Okuyama H., Shimahara Y., Kawada N., The hepatic stellate cell in the post-genomic era., Histol. Histopathol.; 2002, 17(2): 487-495). Hepatitis viruses, alcohol and medicaments generate reactive oxygen species in the liver to induce the apoptosis of hepatocytes, and cause acute hepatitis which may lead to hepatic fibrosis (Shimahara Y., Yamamoto N., Uyama N., Okuyama H., Momoi H., Kamikawa T., Terajima H., Iimuro Y., Yamamoto Y., Ikai I., Kushihata F., Kiyochi H., Kobayashi N., Yamaoka Y., Significance of Serum Type IV Collagen Level of Hepatectomized Patients with Chronic Liver Damage., World J. Surg.; 2002, 26(4): 451-456). The hepatic fibrosis is accompanied by not only the death of hepatocytes but also the activation of hepatic stellate cells. The hepatic stellate cells are activated by radicals and demonstrates various functions, such as proliferation, collagen synthesis, migration and contraction (Kawada N., Kristensen D B., Asahina K., Nakatani K., Minamiyama Y., Seki S., Yoshizato K., Characterization of a stellate cell activation-associated protein (STAP) with peroxidase activity found in rat hepatic stellate cells., J. Biol. Chem.; 2001, 276(27): 25318-25323). These stellate cells greatly participate in hepatic fibrosis. The activation of hepatic stellate cell was suppressed by an antioxidant in a cell culture experiment and hepatic fibrosis was also suppressed by an antioxidant in an animal experiment, showing that oxidative stress is involved with stellate cell activation and hepatic fibrosis.

The inventors have reported that N-acetylcysteine, a glutathione precursor, attenuates thioacetamide-induced hepatic fibrosis (Okuyama H., Shimahara Y., Kawada N., Seki S., Kristensen D B., Yoshizato K., Uyama N., Yamaoka Y., Regulation of cell growth by redox-mediated extracellular proteolysis of platelet-derived growth factor receptor beta., J. Biol. Chem.; 2001, 276(30): 28274-28280). It has been found that ADF expression is enhanced in fetal liver, hepatocellular carcinoma and in the course of hepatic fibrosis. Therefore, ADF is expected to play some role in hepatic diseases.

An object of the present invention is to provide a therapeutic agent for treating hepatic disease comprising thioredoxin as an active ingredient.

### SUMMARY OF THE INVENTION

The present invention relates to a therapeutic agent for treating acute hepatitis comprising one or more polypeptide(s) as active ingredient(s) selected from polypeptides belonging to the family having thioredoxin activity.

The present invention also relates to a. therapeutic agent for treating chronic hepatitis (especially hepatic fibrosis and hepatic cirrhosis) comprising as active ingredient(s) one or more polypeptide(s) selected from those belonging to the family having thioredoxin activity.

### BRIEF DESCRIPTION OF THE DRAWINGS

FIG. 1 shows the expression of thioredoxin in fibrotic liver. Immunohistochemical staining of thioredoxin. Bottom: normal rat liver (100×); top: thioacetamide (TAA)-induced fibrotic liver of rat (100×).

FIG. 2 shows the effects of thioredoxin on acute lethal hepatitis.

2A: survival rate in thioacetamide-induced acute lethal hepatitis (200 μg/g)

2B: survival rate in thioacetamide-induced acute lethal hepatitis (100 μg/g)

2C: serum GOT and GPT levels, 24 hours after thioacetamide administration

2D: histological analysis by hematoxylin eosin staining

2E: detection of apoptosis by TUNEL staining

FIG. 3 shows the effect of thioredoxin on TNF-α-induced cell death in HepG2.

TNF-α and cycloheximide were added to HepG2 stably expressing thioredoxin to induce apoptosis. MTT assay was carried out 24 hours after the addition. EV: pcDNA3 empty vector transfected HepG2. wt: TRX wt stably transfected HepG2. dm: TRX double mutant stably transfected HepG2.

FIG. 4 shows the effects of thioredoxin on hepatic fibrosis.

4A: histological analysis by Mallory Azan staining

4B: evaluation of fibrotic area

4C: serum GOT and GPT levels, 24 hours after the final administration of thioacetamide

FIG. 5 shows the inhibitory effect of thioredoxin on proliferation of cultured stellate cells.

5A: inhibitory effect on the proliferation of stellate cells when cocultured with HepG2 stably expressing thioredoxin

5B: inhibitory effect of thioredoxin on the proliferation ability of stellate cells

### DISCLOSURE OF THE INVENTION

In the specification, a wide range of peptides from oligopeptides to polypeptides (proteins) are included in the polypeptides belonging to the family having thioredoxin activity, insofar as they have thioredoxin activity.

The family having thioredoxin activity contains the sequence of -Cys-X-Y-Cys- in the active site (X and Y represent identical or different amino acids selected from 20 kinds of natural amino acid) and is called thioredoxin superfamily (hereinafter referred to as the “TRX family”).

Examples of the polypeptides belonging to TRX family include polypeptides containing the sequences: -Cys-Gly-Pro-Cys-, -Cys-Pro-Tyr-Cys-, -Cys-Pro-His-Cys- or -Cys-Pro-Pro-Cys- in the active site. Preferred among them are polypeptides having the sequence -Cys-Gly-Pro-Cys- in the active site.

Specifically, polypeptides belonging to the TRX family may comprise thioredoxins of animals including human (ADF of animals including human), thioredoxins of bacteria such as E. coli, thioredoxin of yeasts and like thioredoxins; polypeptides having human ADF activity (human ADFP); glutaredoxins of humans, E. coli and the like.

The preferable polypeptide belonging to the TRX family is thioredoxin, especially human thioredoxin and yeast thioredoxin. Yeast thioredoxin may be isolated from yeast or may be in the form of yeast containing large amounts of thioredoxin.

The polypeptide types belonging to the TRX family can be contained singly or in combination of two or more in the therapeutic agent for treating acute and chronic hepatitis of the present invention.

The polypeptide(s) belonging to the TRX family can be derived from bacteria (E. coli), yeast, plants and animals, especially mammals including human (cows, horses, dogs, cats, monkeys, guinea pigs, rats, mice, rabbits, etc.). In addition, the polypeptide(s) belonging to the TRX family can be obtained from yeast, E. coli and the like by purification of natural products or by gene recombination, and can be a derivative in which one or more, preferably one or a few amino acid(s) are substituted, added or deleted, insofar as TRX activity is remained.

The polypeptide(s) containing thioredoxin can be either an oxidized form or a reduced form, but preferably a reduced form.

Examples of hepatic diseases to be treated are acute hepatitis and chronic hepatitis, which includes viral (acute and chronic) hepatitis and drug-induced (acute and chronic) hepatitis. In particular, hepatic fibrosis and hepatic cirrhosis can be mentioned as examples of chronic hepatitis.

The administration route can be either oral (tablets, coated tablets, powders, granules, capsules, solutions, pills, suspensions, emulsions, syrups, liposome-encapsulated agents, etc.) or parenteral (parenteral injections used for intravenous injection, hypodermic injection, endermic injection, intramuscular injection, intraperitoneal injection and the like, drops, suppositories, ointments, inhalants, nasal drops, etc.).

An effective amount of the polypeptide belonging to the TRX family in a therapeutic agent for treating acute or chronic hepatitis is about 50 to about 500 mg daily for an adult, which may be prescribed in 1 to 3 divided doses per day.

### INDUSTRIAL APPLICABILITY

The present invention can provide a therapeutic agent for acute hepatitis, chronic hepatitis, chronic hepatic fibrosis and hepatic cirrhosis that is free from side effects.

### EXAMPLES

The present invention will be described below in detail with reference to an Example, but is not limited in any way to the Example.

### Example 1

(1) Methods

Materials

Human recombinant thioredoxin was provided by Ajinomoto, Inc. Anti-human thioredoxin antibody was provided by Fujirebio Inc. Collagenase and thioacetamide were purchased from Wako Pure Chemical Industries, Ltd. Pronase E was purchased from Merck Ltd. Antibody against smooth muscle α-actin (α-SMA) was purchased from DAKO A/S. [³H] thymidine and [α-³²P]dCTP were purchased from Amersham Pharmacia.

Animals

Thioredoxin transgenic mice (10 weeks old) were purchased from Oriental Bioservice Inc. C57BL/6 mice (10 week old) were purchased from Japan SLC Inc. Wistar rats were purchased from SLC.

Thioacetamide-Induced Acute Hepatitis Model

Male mice weighing 25-30 g were used for in vivo hepatopathy model. Thioacetamide (200 and 100 μg/g) was injected intraperitoneally into thioredoxin-transgenic mice (n=23) and wild-type mice (n=23). After 24 hours, the mice were anesthetized by diethylether and the livers were removed. A portion of each liver was fixed in 10% formalin and used for histological experiments. The remaining part of the liver tissue was quickly frozen in liquid nitrogen and stored at −80° C. until use.

Thioacetamide-induced chronic hepatic fibrosis model

Thioacetamide (50 μg/g) was injected intraperitoneally three times per week for four weeks into thioredoxin-transgenic mice (n=10) and wild-type mice (n=10). The day after final administration, the mice were anesthetized by diethylether and the livers were removed. A portion of each liver was fixed in 10% formalin and used for histological experiments. The remaining part was quickly frozen in liquid nitrogen and stored at −80° C. until use.

Isolation of Hepatic Stellate Cells of Rat and Mouse

Wistar rat livers and C57BL/6 mouse livers were perfused with pronase E and collagenase, and hepatic stellate cells were isolated using the Nycodenz density gradient centrifugation method. The cell purity was 95% or more as assessed by cell configuration and by detecting vitamin A autofluorescence. The cells were cultured in Dulbecco's Modified Eagle Medium supplemented with 10% serum. The medium was changed every two days.

[³H] Thymidine Uptake

Thioredoxin was added to the culture supernatant of activated stellate cells, and the culture was incubated for 24 hours. Then 1.0 μCi/ml of [³H] thymidine was added thereto and after 6 hours the radioactivity was measured using a scintillation counter.

Western Blotting

The hepatic stellate cells after treated with thioredoxin were homogenized in 1×SDS sample buffer (100 μl/35-mm dish, 62.5 mM Tris-HCl, pH 6.8, 10% glycerol, 2% SDS, 5% 2-β-mercaptoethanol, 1 mM Na₃VO₄) to prepare samples. After heat denaturation, each sample (10 μg) was subjected to SDS-polyacrylamide gel electrophoresis (12%) and then transferred onto an Immobilon P membrane. The membranes were subsequently treated with antibodies against hTRX or α-SMA, reacted with a secondary antibody, and then an ECL detection reagent was applied to detect the band.

Northern Blotting

Total RNA was extracted from the cultured stellate cells using Isogen. The total RNA (20 μg) was separated on 1% agarose gel and transferred onto a nylon membrane (Hybond-N⁺). Double-stranded DNAs amplified by polymerase chain reaction were labeled with [α-³²P]dCTP to prepare probes. The DNA sequences were identified. The employed primers are: α1 typeI collagen, 5′-GATGTCACTGAGACGACGAT-3′ (forward) and 3′-CCTCAAACACCACCTGCAAC-5′ (reverse); glyceraldehyde-3-phosphate dehydrogenase, 5′-ACCACAGTCCATGCCATCAC-3′ (forward) and 3′-TCCACCACCCTGTTGCTGTA-5′ (reverse). Hybridization of the membrane with the probe was carried out and the bands of autoradiography were detected on Kodak XAR5 x-ray films.

Mallory Azan Staining

Tissue samples embedded in paraffin were cut into sections of 4 μm thickness and Azan staining was performed. Mac SCOPE version 2.5 and Photgrab-2500 for Macintosh FUJIX SH-25/M were used to capture the image and the fibrotic area was measured.

Immunohistochemical Staining

The paraffin-embedded tissue sections were treated with 1% hydrogen peroxide, 0.1% proteinase K and 1% Triton X-100. The sections were reacted with mouse monoclonal antibodies against hTRX or α-SMA diluted 50-fold, followed by incubation with biotin-conjugated rabbit anti-mouse IgG F(ab′)₂ diluted 200-fold for 1 hour at room temperature, and colored with DAB.

(2) Results

Thioredoxin Expression in Hepatopathy Liver

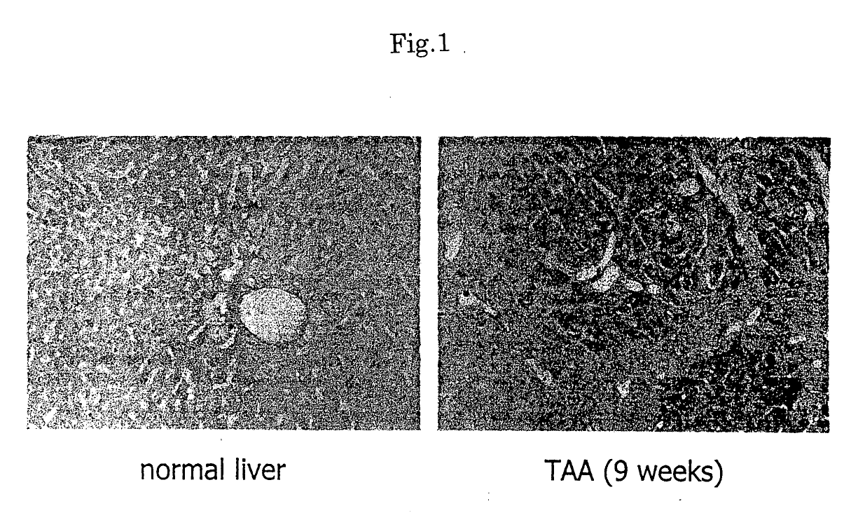

The immunohistochemical staining revealed that the expression of thioredoxin was enhanced in the hepatocytes of chronic hepatic fibrosis but practically undetected in nonparenchymal cells (hepatic stellate cells) located at the fibrotic area. Thioredoxin expression was barely observed in hepatocytes of a normal liver (FIG. 1).

Effect on Thioacetamide-Induced Acute Hepatitis Model

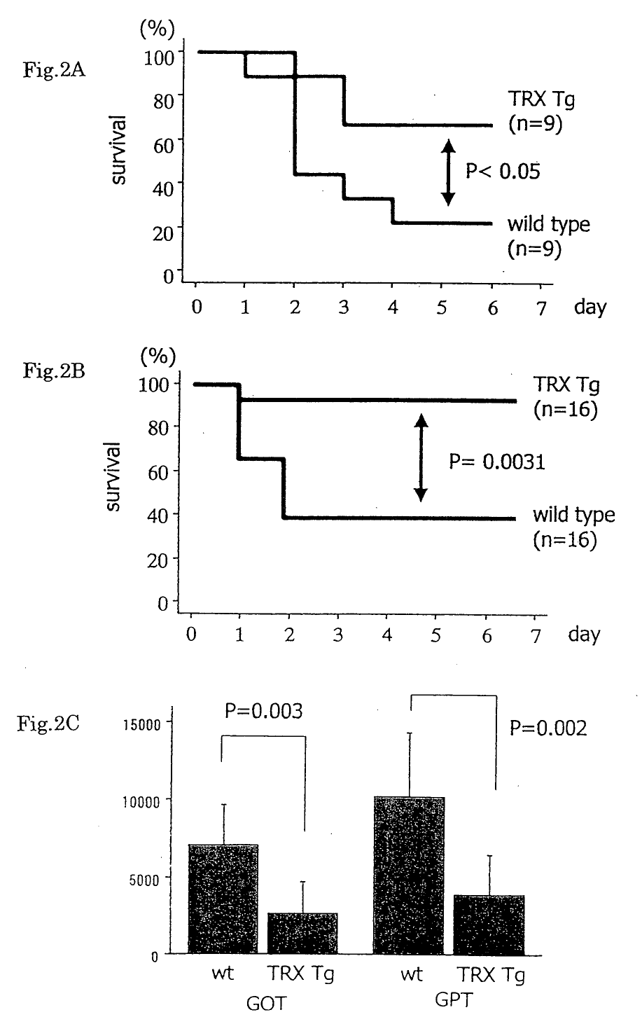

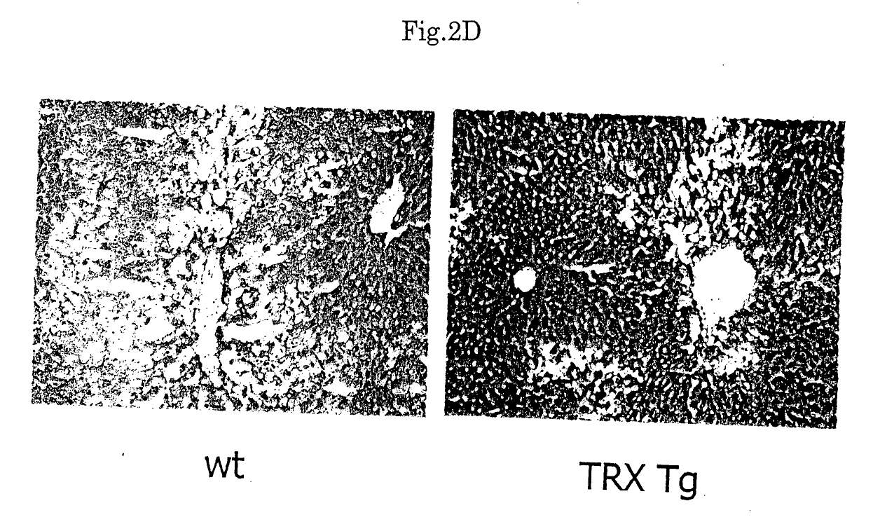

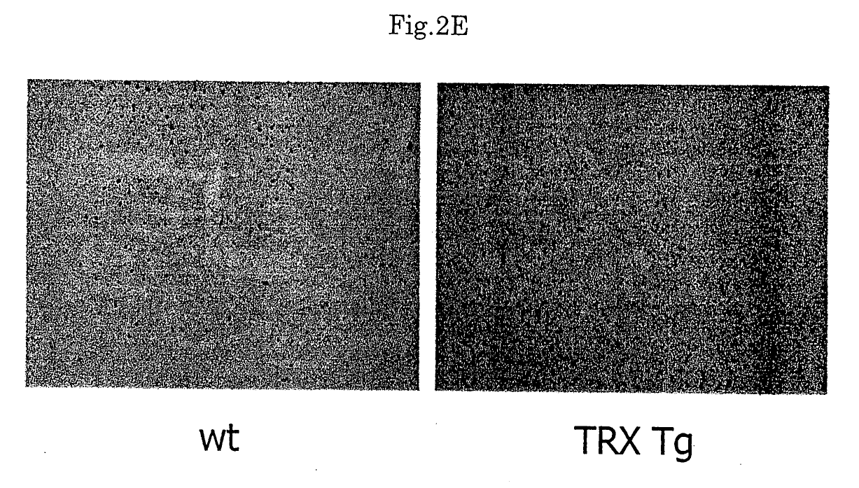

Using thioredoxin-transgenic mice, an acute hepatitis model was applied to study whether thioredoxin is involved in the protection against liver disorder. Wild-type mice and thioredoxin-transgenic mice were subjected to acute hepatitis by intraperitoneal injection of an excessive amount of thioredoxin. The survival rate measured over 7 days after injection was significantly higher in thioredoxin-transgenic mice than in wild-type mice (FIGS. 2A and 2B). The levels of hepatic enzymes AST and ALT were significantly lower in thioredoxin-transgenic mice than in wild-type mice (FIG. 2C). The result of hematoxylin eosin staining showed that the loss of hepatic cells caused by cellular necrosis of hepatic cells was suppressed more in thioredoxin-transgenic mice than in wild-type mice (FIG. 2D).

Suppression of Apoptosis in Thioredoxin-Transgenic Mice

The mechanism of suppression of hepatitis in animal experiments was investigated in relation to apoptosis. As a result of TUNEL staining, the number of TUNEL-positive cells in zone 3 around the central liver veins was significantly smaller in thioredoxin-transgenic mice than in wild-type mice (FIG. 2E).

Suppression of TNF-α-Induced Apoptosis in Hepatic Cell Line HepG2 showing an Enhanced Thioredoxin Expression

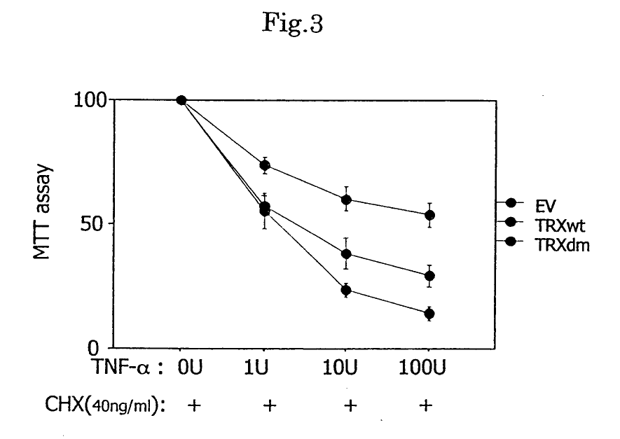

Hepatic cell line HepG2 showing an enhanced expression of thioredoxin was used to study the effect of thioredoxin. Hepatic cell line HepG2 intensively expressing thioredoxin and dominant negative thioredoxin-expressing hepatic cell line HepG2 were established and cultured. Tumor Necrosis Factor-alpha (TNF-α) was added to the culture supernatant to induce apoptosis. The survival rate of the cells was determined by MTT assay. Apoptosis caused by TNF-α was found less in the hepatic cell line HepG2 showing an enhanced thioredoxin expression than in the control hepatic cell line HepG2, and was detected more in the dominant negative thioredoxin-expressing hepatic cell line HepG2 than in the control hepatic cell line HepG2 (FIG. 3).

Effect on Thioacetamide-Induced Chronic Hepatic Fibrosis Model

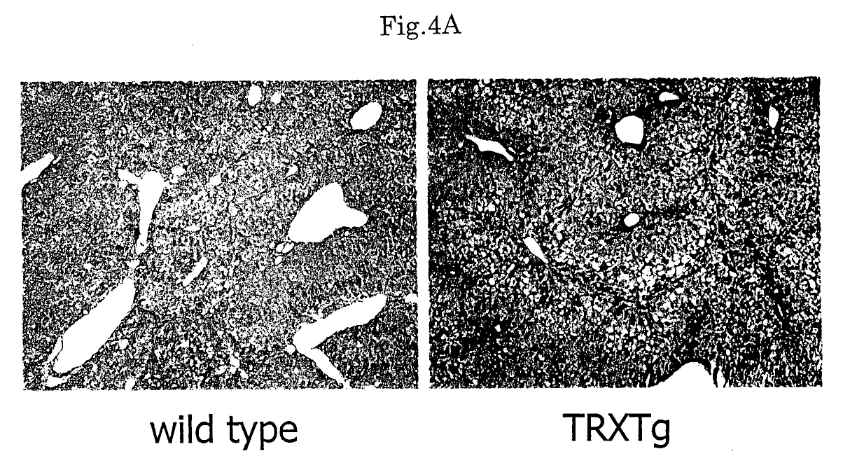

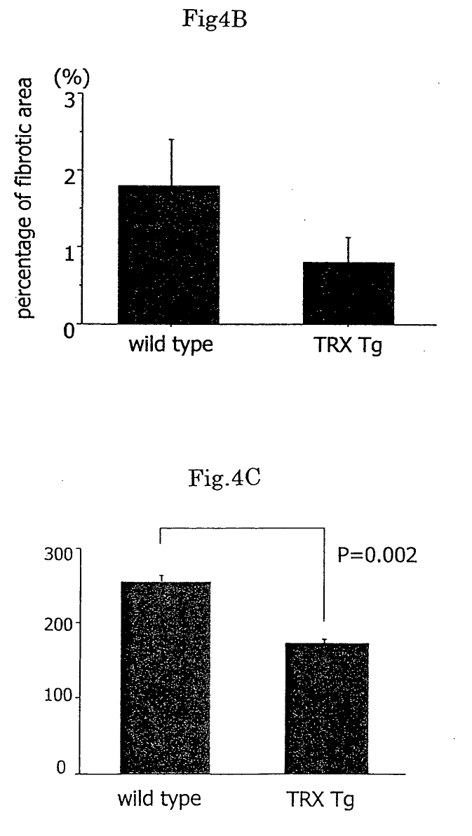

The effect of thioredoxin on hepatic fibrosis was studied. Hepatic fibrosis models were prepared by prolonged administration of thioacetamide, 3 times a week for 4 weeks. Histological observation was carried out by means of Azan staining. The area of hepatic fibrosis region was significantly smaller in thioredoxin-transgenic mice than in wild-type mice (FIGS. 4A and 4B). The levels of hepatic enzymes AST and ALT were significantly lower in thioredoxin-transgenic mice than in wild-type mice (FIG. 4C).

Coculture of Hepatic Cell Line HepG2 Showing an Enhanced Thioredoxin Expression with Primary-Cultured Stellate Cells

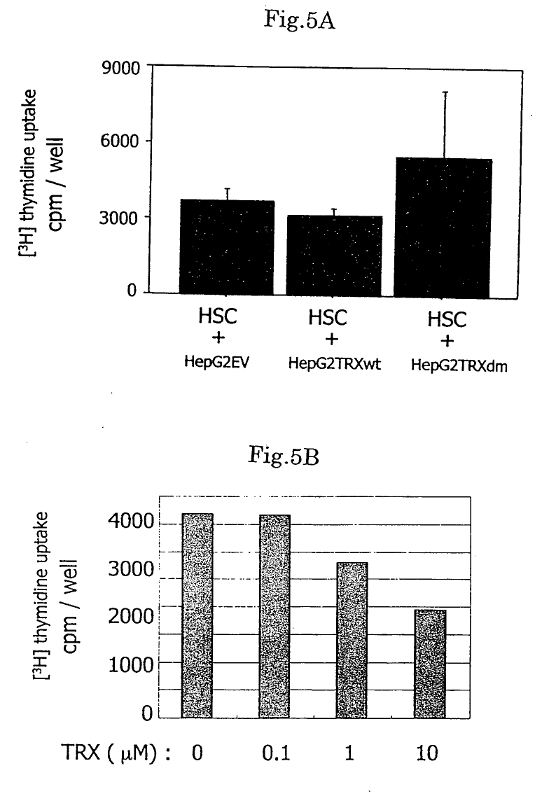

For the purpose of investigating whether hepatic stellate cells are involved in the suppression of hepatic fibrosis in thioredoxin-transgenic mice, the primary-cultured hepatic stellate cells isolated and cultured from rat livers were cocultured with the hepatic cell line HepG2 intensively expressing thioredoxin or with the dominant negative thioredoxin-expressing hepatic cell line HepG2. DNA synthesis ability of hepatic stellate cells after 24 hours of coculture was analyzed by [³H] thymidine uptake experiments. The hepatic stellate cells cocultured with the hepatic cell line HepG2 intensively expressing thioredoxin showed lower DNA synthesis ability than those cocultured with control hepatic cell line HepG2, and the hepatic stellate cells cocultured with the dominant negative thioredoxin-expressing hepatic cell line HepG2 showed higher DNA synthesis ability than those cocultured with control hepatic cell line HepG2 (FIG. 5A). These results suggest that thioredoxin produced by the hepatic cell line HepG2 might inhibit the proliferation ability of activated hepatic stellate cells.

Inhibitory Effect of Exogenous Thioredoxin on the Proliferation of Cultured Stellate Cells

The inhibitory effect of thioredoxin on the proliferation ability of hepatic stellate cells was examined. Recombinant thioredoxin was added to a culture supernatant of cultured stellate cells and the DNA synthesis ability of hepatic stellate cells was analyzed by [³H] thymidine uptake experiments. The results show recombinant thioredoxin inhibits the proliferation of hepatic stellate cells depending on its concentration (FIG. 5B).

(3) Discussion

In the Example, the effect of thioredoxin on acute hepatitis and chronic hepatic fibrosis was studied using thioredoxin-transgenic mice. In the thioacetamide-induced acute hepatitis model, improved results were obtained as to survival rates, hepatic enzymes and tissues of thioredoxin-transgenic mice. In addition, TUNEL staining showed that apoptosis was suppressed in thioredoxin-transgenic mice. Thioacetamide is known to damage hepatocytes by generating reactive oxygen species in the cytoplasm thereof. Reactive oxygen species also induce apoptosis of hepatocytes resulting in cell death. From the above facts it is considered that the anti-apoptosis effect of thioredoxin in the thioacetamide-induced acute hepatitis model is ascribable to the elimination of reactive oxygen species. In thioredoxin-transgenic mice of the cerebral ischemia model, it was shown that carbonylation of protein is inhibited after middle cerebral artery occlusion and the symptoms of cerebral ischemia can be improved. The present inventors have previously reported that cells intensively expressing thioredoxin show resistive activity against peroxynitrite-induced cytotoxicity. An anti-apoptosis mechanism of thioredoxin has also been reported. Thioredoxin suppressed p53 and p38 to inhibit apoptosis, and blocked ASK-1 to inhibit TNF-α-induced apoptosis.

Hepatic fibrosis has also been improved in thioredoxin-transgenic mice. Since the hepatic enzyme levels were significantly decreased, it is considered that the anti-fibrosis effect of thioredoxin may be incidental to the inhibitory effect on hepatic cell death. Interestingly however, exogenous thioredoxin was found to show an inhibitory effect on hepatic stellate proliferation. In fact, DNA synthesis of hepatic stellate cells was inhibited by the coculture with hepatic cell line HepG2 that shows an enhanced thioredoxin expression (Uyama N., Shimahara Y., Kawada N., Seki S., Okuyama H., Iimuro Y., Yamaoka Y., Regulation of cultured hepatocyte proliferation by stellate cells., J. Hepatol.; 2002, 36(5): 590-599). These results suggest that thioredoxin, the expression of which is enhanced in hepatocytes of hepatic fibrosis, not only has an anti-apoptosis effect on hepatocytes but also an anti-proliferation effect on peripheral hepatic stellate cells.

We have previously reported that the antioxidant N-acetylcysteine inhibits platelet-derived growth factor (PDGF)-dependent proliferation of hepatic stellate cells. The mechanism of the inhibition was based on the finding that N-acetylcysteine enhances the enzyme activity of cathepsin B secreted from stellate cells to degrade the PDGF receptor (Kristensen D B., Kawada N., Imamura K., Miyamoto Y., Tateno C., Seki S., Yoshizato K., Proteome analysis of rat hepatic stellate cells., Hepatology.; 2000, 32(2): 268-277). In this study, thioredoxin also showed no effect on cell membrane receptors while inhibiting the proliferation of hepatic stellate cells. Since oxidative stress contributes to the activation of hepatic stellate cells including proliferation, the anti-proliferation effect of thioredoxin is considered to be based on the elimination of reactive oxygen species.

It is well known that fibrosis is a major function of activated stellate cells, similar to proliferation (Kawada N., Tran-Thi T A., Klein H., Decker K., The contraction of hepatic stellate (Ito) cells stimulated with vasoactive substances. Possible involvement of endothelin-1 and nitric oxide in the regulation of the sinusoidal tonus., Eur. J. Biochem.; 1993, 213(2): 815-823). Anti-fibrosis treatment can be an effective remedy against hepatic cirrhosis (Okuno M., Akita K., Moriwaki H., Kawada N., Ikeda K., Kaneda K., Suzuki Y., Kojima S., Prevention of rat hepatic fibrosis by the protease inhibitor, camostat mesilate, via reduced generation of active TGF-beta., Gastroenterology.; 2001, 120(7): 1784-1800). As previously reported by the present inventors, the antioxidant N-acetylcysteine inhibits collagen synthesis at mRNA level not only in animal models but also in cultured stellate cells. In the case of thioredoxin, however, it showed the tendency of promoting collagen synthesis in cultured hepatic stellate cells. In addition, mouse fibroblast cell line NIH3T3 showing an enhanced thioredoxin expression was prepared and the relationship between collagen synthesis and thioredoxin was examined, with results that thioredoxin showed no effect on collagen synthesis in NIH3T3 (data unpublished). These results show that the inhibitory effect of thioredoxin on fibrosis in animal models is based on the effects of inhibiting the apoptosis of hepatocytes and suppressing the proliferation of stellate cells.

Some therapeutic drugs for fibrosis have been heretofore reported. However, none of these drugs have been easily applied to clinical use, since they are artificial and beset by the problems of side effect. In contrast, thioredoxin is an endogenous thiol-containing protein which can be expressed internally and therefore is considered more adaptable to clinical use.

In conclusion, it is confirmed that thioredoxin can be an effective remedy against acute and chronic hepatic diseases.

1. A therapeutic agent for treating hepatic disease comprising one or more polypeptide or polypeptides belonging to a thioredoxin family as active ingredient.

2. The therapeutic agent according to claim 1, wherein the hepatic disease is acute hepatitis, chronic hepatitis, chronic hepatic fibrosis or hepatic cirrhosis.

3. The therapeutic agent according to claim 1, wherein the polypeptide belonging to the thioredoxin family is human thioredoxin.

4. The therapeutic agent according to claim 1 in the form of parenteral or oral agent.

5. A method for treating hepatic disease comprising administering an effective amount of one or more polypeptide(s) belonging to the thioredoxin family to a patient in need of such treatment, wherein the hepatic disease is acute hepatitis, chronic hepatitis, chronic hepatic fibrosis or hepatic cirrhosis.

6. The method according to claim 5, wherein about 50 to about 500 mg of thioredoxin is administered to an adult per day.


SEQUENCE LISTING (4 sequences)

SEQ ID NO: 1 - DNA, 20 bp, artificial sequence
  Sequence 1 is an ?1 typeI collagen forward primer.
gatgtcactgagacgacgat

SEQ ID NO: 2 - DNA, 20 bp, artificial sequence
  Sequence 2 is an ?1 typeI collagen reverse primer.
cctcaaacaccacctgcaac

SEQ ID NO: 3 - DNA, 20 bp, artificial sequence
  Sequence 3 is a glyceraldehyde-3-phosphate dehydrogenase forward primer.
accacagtccatgccatcac

SEQ ID NO: 4 - DNA, 20 bp, artificial sequence
  Sequence 4 is a glyceraldehyde-3-phosphate dehydrogenase reverse primer.
tccaccaccctgttgctgta